(19) United States
(12) Patent Application Publication
(10) Pub. No.: US 2025/0362176 A1
(43) Pub. Date: Nov. 27, 2025

(54) SENSOR MODULE FOR RAMAN SPECTROSCOPY, ELECTRONIC DEVICE AND METHOD OF CONDUCTING RAMAN SPECTROSCOPY

(71) Applicant: ams International AG, Jona (CH)

(72) Inventors: Rainer MINIXHOFER, PREMSTÄTTEN (AT); Jochen KRAFT, BRUCK AN DER MUR (AT); Remco VERDOOLD, GELDROP (NL); Tanja KOCH, Bruckmühl (DE)

(21) Appl. No.: 18/993,075

(22) PCT Filed: Jun. 23, 2023
(86) PCT No.: PCT/EP2023/067144

§ 371 Date: Jan. 10, 2025

(30) Foreign Application Priority Data
Jul. 12, 2022 (DE) 10 2022 117 346.1

(51) Int. Cl. G01J 3/44; G01J 3/02; G01J 3/10; G01J 3/18

(52) U.S. Cl. CPC G01J 3/44; G01J 3/0259; G01J 3/0272; G01J 3/10; G01J 3/1895

(57) ABSTRACT

A sensor module for Raman spectroscopy includes a sensor package enclosing a light emitter arrangement, a dispersive element and a light detector arrangement arranged on or integrated into a carrier. The light emitter arrangement is operable to emit light with multiple excitation wavelengths out of the sensor module. The dispersive element is operable to receive light incident on the sensor module and operable to disperse the incident light into spectral components. The light detector arrangement is operable to generate spectral sensor signals indicative of the spectral components.

This disclosure relates to a sensor module for Raman spectroscopy, an electronic device and a method of conducting Raman spectroscopy.

### BACKGROUND

The detection of skin constituents, especially blood glucose, has been a challenging task for decades. The technical problem of skin constituent measurements relates to the fact that only a very small optical signal is reflected back from the skin, when illuminated by a light source. Furthermore the diffusive properties of turbid media like skin further scatter the signal. The biological molecules like Urea, Lactate and Glucose have their strongest optical “fingerprint” in the long wavelength range between 8 μm to 12 μm, where limited emitter and detector technologies are available. However, the skin is highly absorbing in these wavelength regions due to water absorption and thus the penetration depth of optical radiation is too shallow to reach the deeper layers of the skin with the constituents of interest.

Raman spectroscopy relies on inelastic light scattering, where a photon excites the sample (Raman Effect). The laser light interacts with molecular vibrations, phonons or other excitations in the system, resulting in the energy of the laser photons being shifted up or down. The light excites a molecule into a virtual energy state for a short time before a photon is emitted. The shift in energy gives information about the vibrational modes in the system. From a technological standpoint, Raman spectroscopy allows to use laser diodes, which are readily available in the visible or near infrared, for example.

Lately there has been a tremendous advancement in the field of photonics. Thanks to photonics Raman spectroscopy has become much more accessible to users in various fields. Footprints of complete spectrometers can be further miniaturized with the help of compact integrated optics, optoelectronics and laser sources. However, Raman spectrometer still remain research-grade instrumentation and benchtop system.

There is a growing need for mobile devices such as mature spectrometers in Smart Watches, Medical and point-of-care or other handheld devices. Bio-sensing application to detect skin constituents like Urea, Lactate and interstitial fluid or blood Glucose is only one example. Compact handheld devices are expected to have a huge impact on most potential spectrometer applications, e.g. material analysis, environmental analysis, biosensors, Smart Health, Medsumer, Point-of-Care, Medical etc.

Thus, an object to be achieved is to provide a sensor module for electronic devices that overcomes the aforementioned limitations and provides compact means to conduct Raman spectroscopy in handheld devices. A further object is to provide an electronic device comprising such a sensor module and a method of conducting Raman spectroscopy.

These objectives are achieved with the subject-matter of the independent claims. Further developments and embodiments are described in dependent claims.

### SUMMARY OF THE DISCLOSURE

The following relates to an improved concept in the field of Raman spectroscopy. One aspect relates to a sensor module which combines two or more light emitters together with a highly dispersive element and a single photon detector, e.g. within a closed loop control scheme of lock-in-amplification. The two or more light emitters could be replaced with a tuneable laser source as well, increasing the spectral bandwidth further. The proposed concept allows for miniaturization and cost of goods sold (COGS) reduction of a Raman spectrometer to enable implementation into mobile or handheld devices.

In at least one embodiment a sensor module for Raman spectroscopy comprises a sensor package enclosing a light emitter arrangement, a dispersive element and a light detector arrangement arranged on or integrated into a carrier.

The light emitter arrangement is operable to emit light with multiple excitation wavelengths out of the sensor module. The dispersive element is operable to receive light incident on the sensor module and operable to disperse the incident light into spectral components. The light detector arrangement is operable to generate spectral sensor signals indicative of the spectral components.

In order to obtain a desired spectral range the dispersive element usually needs to provide a certain bandwidth and resolution. These parameters have an impact on how large the dispersive element needs to be. Often the requirements for accuracy and desired spectral range contradict the possibility to use the dispersive element in a handheld device. The proposed module employs multiple excitation wavelengths in order to essentially extend the spectral range with the dispersive element. In turn, the dispersive element can be relaxed and smaller elements can be implemented inti handheld devices, while still allowing accurate measurements.

This way, a miniaturized Raman Spectrometer can be created fitting into a footprint of about 1 cm². It employs multiple light sources and one single dispersive element (e.g., an arrayed waveguide grating) to generate a broader spectral dataset to cover the molecular fingerprints of analytes, such as Lactate, Urea and Glucose for identification and quantitative detection of molecular concentrations. Broadening of the spectral detection range can be achieved by different excitation lasers together with a high resolution dispersive element. Use of very high sensitive detectors (like SPADs or PIN diodes) further extends detection also to very low signal from the skin. Implementation of advanced control algorithms like lock-in-amplification may further boost the signal-to-noise ratio.

In at least one embodiment, the dispersive element is operable to disperse the incident light into spatially separate spectral components. The spatially separate spectral components can be detected by an array of light emitters, for example or spaced apart single detectors.

In at least one embodiment, the dispersive element comprises one of an arrayed waveguide grating, a diffraction grating, a refractive prism, and/or a poled domain prism.

In at least one embodiment, the light emitter arrangement comprises two or more light emitter, each operable to emit light out of the sensor module with an excitation wavelength from the multiple excitation wavelengths. Providing the excitation wavelengths can be done using single light emitters, which are driven by a driver circuit, for example. This way, the target may be excited with one excitation wavelength at a time.

In at least one embodiment, the light emitter arrangement comprises at least one tuneable light emitter operable to emit light out of the sensor module to be tuned to an excitation wavelength from the multiple excitation wavelengths. Providing the excitation wavelengths can be done by tuning a single light emitter to a desired wavelength e.g. by a driver circuit, for example. This way, the target may be excited with one excitation wavelength at a time.

In at least one embodiment, the light detector arrangement comprises an array of light detectors, each operable to generate one of the spectral sensor signal indicative of a respective spectral component. This way, the target may be excited with one excitation wavelength at a time or with several excitation wavelength at a time.

In at least one embodiment, the carrier comprises a photonic integrated circuit. The PIC allows to integrate photonic functions, e.g. input, dispersion and output of optical fields, e.g. the incident light. This allows to further reduce footprint of the sensor module.

In at least one embodiment, the photonic integrated circuit comprises a single input port to receive the incident light, the input port being an input waveguide. Furthermore, the photonic integrated circuit comprises at least one output port, the output port being an output waveguide. The dispersive element is arranged between the input waveguide and the output waveguide on the photonic integrated circuit chip. The output port is operable to couple the spectral components from dispersive element to the light emitter arrangement.

In at least one embodiment, the input port comprises one of a grating fabricated on a surface of the photonic integrated circuit, a tapered waveguide at the edge of the carrier, a refractive lens, a diffractive lens, and/or an optical fiber.

In at least one embodiment, the output port comprises one of an array of waveguides, each waveguide located at specific spatial locations to couple the spectral components from dispersive element, a single waveguide, multiple waveguides with different widths, and/or multiple waveguides with a specific spacing between individual waveguides.

In at least one embodiment, the sensor package comprises a hollow housing. The housing comprises a first aperture to allow the light emitted by the light emitter arrangement to leave the sensor module and the housing further comprises a second aperture to allow incident light to enter the sensor module.

The housing can be molded and manufactured at a wafer-level. It provides a solid frame to define optical paths and mount of the components of the sensor module. This way, the module can be embedded into an electronic device, such as a handheld device, for example.

In at least one embodiment, the housing comprises optically isolated first and second chambers. The first chamber encloses the light emitter arrangement. The second chamber encloses the light detector arrangement.

In at least one embodiment, the module fits into a footprint of about 1 cm².

In at least one embodiment, an electronic device comprising a sensor module for Raman spectroscopy according to one of the aforementioned aspects and a host system. The sensor module is embedded in and electrically connected to the host system. The host system comprises one of a mobile device, Smartphone, handheld computer, Smart Watch, handheld Medical-device, or a point-of-care device. Possible applications include In-line production quality assessment (or even perhaps gender selection of embryos in poultry eggs).

Furthermore, a method of conducting Raman spectroscopy is suggested, using a sensor module comprising a sensor package enclosing a light emitter arrangement, a dispersive element and a light detector arrangement arranged on or integrated into a carrier.

The method comprises the step of, using the light emitter arrangement, emitting light with multiple excitation wavelengths out of the sensor module. Another step involves, using the dispersive element, receiving light incident on the sensor module and dispersing the incident light into spectral components. Another step involves, using the light detector arrangement, generating spectral sensor signals indicative of the spectral components.

Further embodiments of the method become apparent to the skilled reader from the aforementioned embodiments of the sensor module and of the electronic device, and vice-versa.

### BRIEF DESCRIPTION OF THE DRAWINGS

The following description of figures may further illustrate and explain aspects of the sensor module for Raman spectroscopy, electronic device and the method of conducting Raman spectroscopy. Components and parts of the sensor module that are functionally identical or have an identical effect are denoted by identical reference symbols. Identical or effectively identical components and parts might be described only with respect to the figures where they occur first. Their description is not necessarily repeated in successive figures.

In the figures:

FIG. 1 shows an example embodiment of a sensor module for Raman spectroscopy,

FIG. 2 shows an example embodiment of the emitter section,

FIG. 3 shows an example flow-chart of conducting Raman spectroscopy with the proposed sensor module,

FIG. 4 shows a more detailed example flow-chart of conducting Raman spectroscopy with the proposed sensor module,

FIG. 5 shows another more detailed example flow-chart of conducting Raman spectroscopy with the proposed sensor module,

FIG. 6 shows an example application of the proposed sensor module, and

FIG. 7 shows an example scheme of illumination geometry.

### DETAILED DESCRIPTION

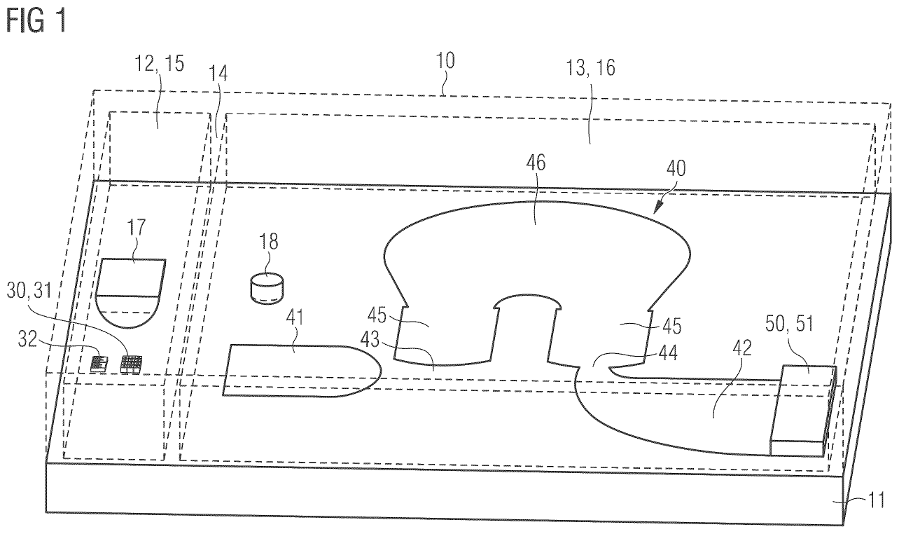

FIG. 1 shows an example embodiment of a sensor module for Raman spectroscopy. The drawings shows a schematic of the sensor module comprising a sensor package with a housing 10 arranged on a carrier 11. The sensor package encloses a light emitter arrangement 30, a dispersive element 40 and a light detector arrangement 50 arranged on or integrated into a carrier. The housing can be molded and comprises a hollow molded body which is mounted on the carrier. Furthermore, the light emitter arrangement and the semiconductor light detector arrangement are placed behind respective apertures to emit light out of the sensor module and receive incident light. The housing can be arranged with chambers, a first chamber 12 encloses the light emitter arrangement and a second chamber 13 encloses the light detector arrangement and the dispersive element. The chambers are separated by a wall 14, e.g. of opaque mold material, of the housing to optically isolate the light emitter arrangement from the dispersive element and the light detector arrangement. This way, the housing can be considered to have an emitter section 15 and a spectrometer section 16.

The carrier 11 can be complemented with or can be arranged as a photonic integrated circuit, PIC for short. The light emitter arrangement 30, dispersive element 40 and light detector arrangement 50 can be arranged on or integrated into the PIC. In this example, the PIC comprises an input port 41 to receive the incident light. The input port is implemented as an input waveguide, e.g. with a grating coupler. Furthermore, the PIC comprises an output port. An output port 42 is implemented as an output waveguide, e.g. with a grating coupler.

Furthermore, the dispersive element 40 is integrated into the PIC and arranged between the input waveguide 41 and output waveguide 42 on the photonic integrated circuit chip. The dispersive element comprises an arrayed waveguide grating, or AWG. The AWG comprises a planar waveguide fabricated by depositing doped and undoped layers of silica on the PIC, based on a silicon substrate. An input side 43 of the AWG is optically coupled to the input port to couple incident light into the structure. An output side 44 of the AWG is optically coupled to the output port 42. Furthermore, the AWG comprises free space propagation regions 45 and a plurality of grating waveguides 46. Typically, the grating waveguides have a constant length increment and form channels of AWG. The AWG could also be done with other waveguide materials than the currently proposed silica-based or SiN waveguides. Materials include electro-optical materials like BTO, LNO, then also the AWG could be tuned to a variety of bandwidths. For example, the AWG has a spectral bandwidth of 20 nm and 0.2 nm per channel.

The emitter section 15 comprises a first aperture 17. The aperture is arranged such that light emitted by the light emitter arrangement 30 can leave the sensor module, e.g. to be directed to an external target (not shown) and to excite a probe or specimen therein. The first aperture 17 can be complemented with an exit lens, e.g. a cylinder lens.

The light emitter arrangement 30 comprises one or more semiconductor light emitters 31, such as semiconductor laser diodes and/or resonant cavity light-emitting devices. Each is operable to emit light out of the sensor module with an excitation wavelength, thus forming a set of multiple excitation wavelengths. The light emitters feature coherent emission to generate light at various excitation wavelengths. A resonant-cavity light emitting device can be considered a semiconductor device, which is operable to emit coherent light based on a resonance process. In this process, the resonant-cavity light emitting device may directly convert electrical energy into light, e.g., when pumped directly with an electrical current to create amplified spontaneous emission. However, instead of producing stimulated emission only spontaneous emission may result, e.g., spontaneous emission perpendicular to a surface of the semiconductor is amplified.

One example relates to the vertical cavity surface emitting laser, VCSEL, diodes. VCSELs are an example of a resonant-cavity light emitting device and feature a beam emission that is perpendicular to a main extension plane of a top surface of the VCSEL. The VCSEL diode can be formed from semiconductor layers on a substrate, wherein the semiconductor layers comprise distributed Bragg reflectors enclosing active region layers in between and thus forming a cavity. VCSELs and their principle of operation are a well-known concept and are not further detailed throughout this disclosure. For example, there may be an array of VCSEL diodes configured to have emission wavelengths of 830 nm, 840 nm and 850 nm, or another natural wavelength, e.g. 785 nm.

However, there may only be a single semiconductor light emitter 31 forming the light emitter arrangement 30 Then the light emitter is tuneable to emit light out of the sensor module with a tuned excitation wavelength from the multiple excitation wavelengths. VCSELs, or other surface or edge emitting laser diodes can also be tuneable and driven to emit at various wavelengths.

The spectrometer section 16 comprises a second aperture 18. The aperture is arranged such that incident light, e.g. emitted by the external target due to Raman scattering, can enter the spectrometer section and be coupled into the dispersive element 40, e.g. by way of the input port 41. The second aperture 18 can be complemented with an entrance lens. For example, the entrance pupil lens of the spectrometer has an assumed focal length of about 1 mm.

The light detector arrangement comprises an array of semiconductor light detectors 51, such as photon counters, e.g. single photon avalanche diodes, or SPADs, Avalanche Photo Diodes, or APDs, Silicon photomultipliers, or SiPMs, semiconductor photodiodes or charge coupled devices, or CCDs, or MEMS photo multipliers, or PMs. For example, a silicon based photodiode array (e.g. SPADs) are complemented with bandpass filters, such as 900 to 930 nm bandpass filters. A number of light detectors in the array matches the number of channels of the AWG. The light detectors can be flip-chip bonded to carrier or alternative integrated into the PIC.

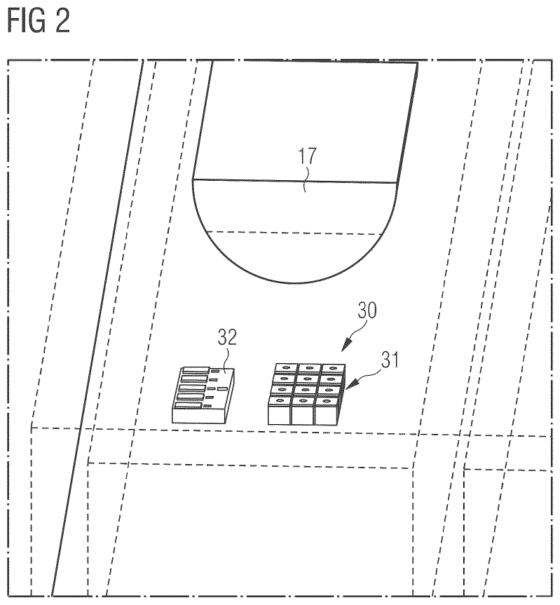

FIG. 2 shows an example embodiment of the emitter section. This drawing shows a more detailed view of the emitter section 15. The exit lens in the first aperture 17, e.g. cylinder lens, has a focus length of about 1 mm and the external target is assumed to be placed outside the sensor module in the focal plane of the exit lens to achieve best excitation. For example, the exit lens focuses three VCSEL-type light emitters on one section of the external target, e.g. Human skin. An optional wavelength detector 32 can be provided to monitor laser emission wavelengths and enable active emission wavelength stabilization. In a simple setup the laser emission wavelength can be stable enough to ensure sufficient accuracy of the spectrum acquisition. In this example, the array of light emitters comprises three 10 nm spaced VCSEL diodes emitting at 850, 840, and 830 nm. Further array may be implemented to improve illumination, e.g. four columns of three-VCSEL arrays for four section illumination of skin.

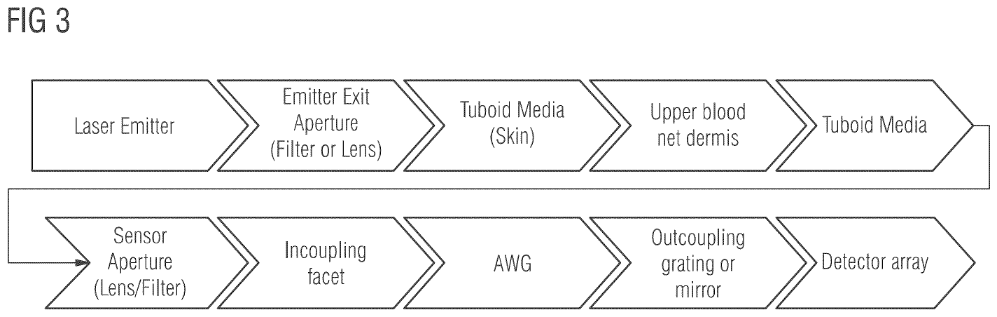

FIG. 3 shows an example flow-chart of conducting Raman spectroscopy with the proposed sensor module. In operation, the sensor module is placed on the external target. One desired external target is human skin to detect Lactate, Urea and Glucose Spectral Footprints noninvasively.

Briefly, light is emitted with one of multiple excitation wavelengths. The excitation light leaves the sensor module via the exit lens and is directed towards the external target, e.g. turbid media (skin). For example, the excitation light eventually reaches the areas in the skin where interstitial fluid containing the skin constituents of interest is present (below the Stratum Corneum, the upper epidermis) or deeper in the skin where sufficient blood is already present, e.g. the upper blood net dermis. At the external target the excitation light undergoes Raman scattering and, in part, is scattered back towards the sensor module, e.g. via the turbid media. Eventually, the back scattered light is incident on the sensor module and enters the entrance lens. This incident light is then coupled into the AWG by way of the input port. The AWG disperses the incident light into spectral components, which are then coupled out by way of the output port and directed to the light detector arrangement, which, in turn, generates spectral sensor signals indicative of the spectral components.

In more detail, incident light is coupled into the AWG via the input waveguide of the input port. Light diffracting out of the input waveguide propagates through the free-space region and illuminates the grating with a Gaussian distribution. Each wavelength of light coupled to the grating waveguides undergoes a constant change of phase attributed to the constant length increment in grating waveguides. Light diffracted from each waveguide of the grating interferes constructively and gets refocused at the output waveguides of the output port, with the spatial position, the output channels, being wavelength dependent on the array phase shift.

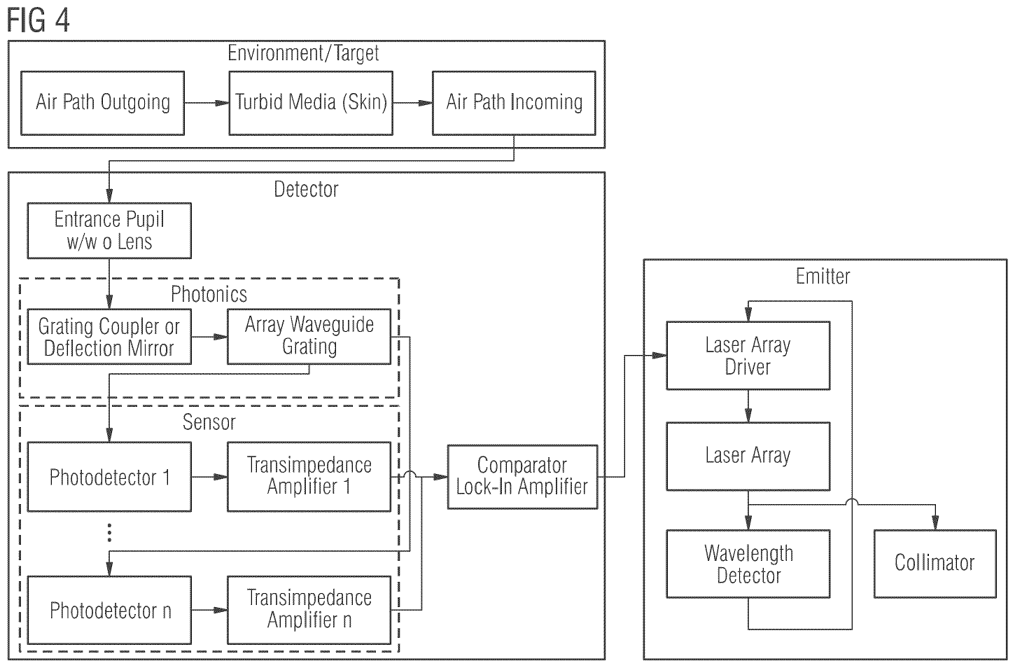

FIG. 4 shows a more detailed example flow-chart of conducting Raman spectroscopy with the proposed sensor module. The chart illustrates the environment including the external target, the emitter section and the spectrometer section.

The emitter section 15 comprises a laser array driver, which drives the array 31 of light emitters to emit light with an excitation wavelength. Optionally, the wavelength detector monitors emission wavelengths of the array and enable active emission wavelength stabilization via a feedback to the laser array driver. The emitted light leaves the emitter section via the exit lens and is directed to the environment including the external target.

The environment including the external target is represented in the chart as an air path outgoing, the target, e.g. turbid media of human skin, and air path incoming. As a result incident light of a particular excitation wavelength excited the target and undergoes Raman scattering. Eventually, the back scattered light is incident on the sensor module and enters the spectrometer section.

The spectrometer section 16 comprises the entrance lens to receive the Raman scattered incident light. The Raman scattered incident light is coupled into the PIC by way of the input port, e.g. a grating coupler or deflection mirror and further into the AWG. Dispersed light leaves the AWG via the output port and is incident on the array of light detectors. The light detectors are complemented with respective transimpedance amplifiers to amplify spectral sensor signals generated by the light detectors and provide respective output signals. The output signals are provided to a comparator and lock-in amplifier to extract spectral output signals from the potentially extremely noisy output signals. These signals provide the spectral output of the sensor module and are also fed back to the laser array driver to establish a closed loop control scheme of lock-in-amplification.

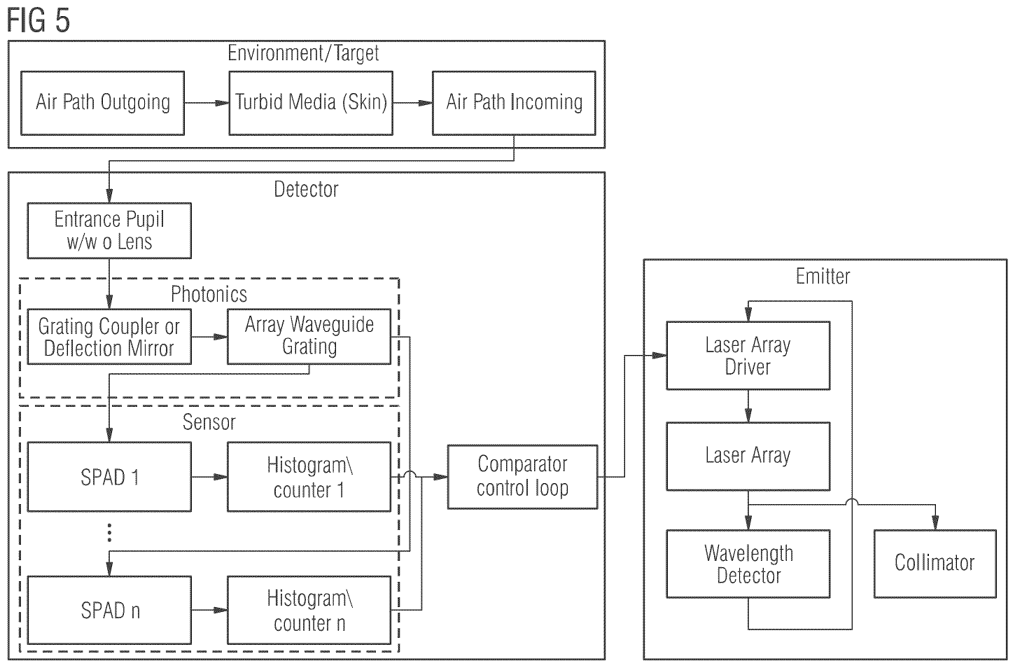

FIG. 5 shows another more detailed example flow-chart of conducting Raman spectroscopy with the proposed sensor module. In this case, an array of SPADs is used as used as light detectors. Instead of respective transimpedance amplifiers, SPADs involve respective histogram and counters and provide respective output signals. The output signals are provided to a comparator control loop to extract spectral output signals from the potentially extremely noisy output signals. These signals provide the spectral output of the sensor module and are also fed back to the laser array driver to establish a closed loop control scheme.

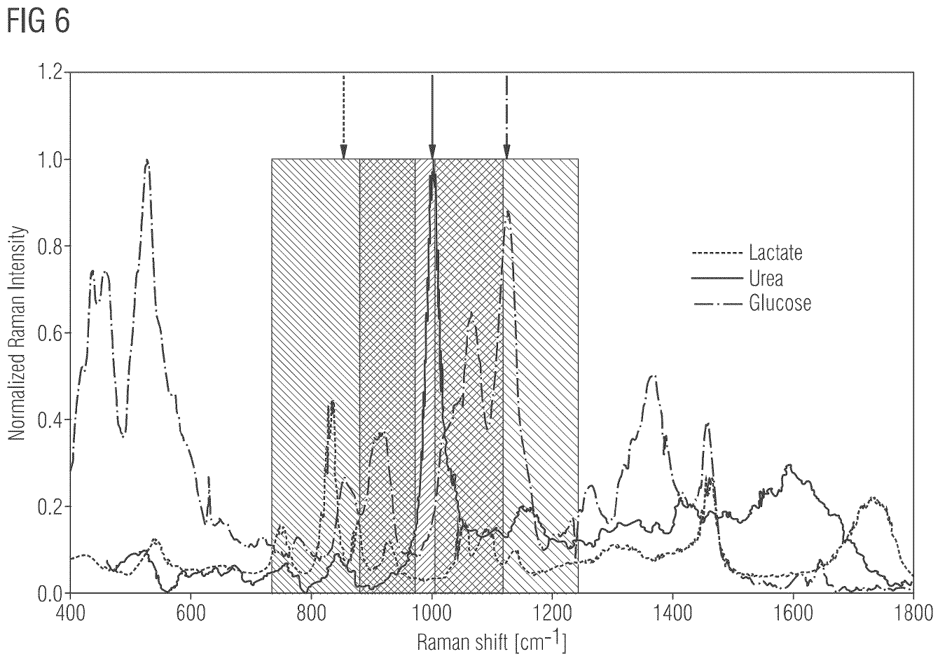

The charts of FIGS. 4 and 5 are illustrated based on a given excitation wavelength. The emitter section 15 can in the same way be driven to emit also other excitation wavelengths from a set of multiple excitation wavelengths. Using essentially the same component, the spectral range can be extended as will be discussed in FIG. 6.

FIG. 6 shows an example application of the proposed sensor module. The graph shows Raman spectra of Lactate, Urea and Glucose. Main Raman peaks of the analytes are found at 854 cm⁻1, 1000 cm⁻¹ and 1125 cm⁻¹ and have an FWHM of about 20 cm⁻¹. Furthermore, the graph also shows areas representing AWG spectral ranges for three different excitation wavelength of the light emitter arrangement 30.

For example, a 100 channel AWG with a very high resolution of 0.2 nm per channel is selected to resolve the required spectral features of the example analytes. One single center Raman shifted wavelength of 916 nm for the AWG is chosen. Because of the high etendue of a single channel in an AWG this spectral resolution is feasible. However, due to size constraints (AWG gets very large in terms of surface area) and optical power constraints (power is distributed over the number of channels) the spectral bandwidth is limited and in this example needs to be limited to 20 nm. When the sensor module is implemented into a handheld or mobile device, multiplying the number of AWGs is out of scope due to their large areas.

With the proposed sensor module different excitation wavelengths can be used instead of larger AWGs to excite Raman scattering in the example analytes. In a certain sense multiplying the number of light sources is proposed to compensate for the difference in Raman peaks of the three analytes and to extend the spectral range despite a smaller AWG. Based on the AWG specifications discussed above and the position of the Raman peaks of the example analytes center wavelengths are found to be at 849.5 nm, 839.1 nm and 830.4 nm, respectively. The excitation wavelengths of an array of VCSELs can be set emission wavelengths of 830 nm, 840 nm and 850 nm specified laser. This way the main peaks of the example analytes are covered nicely. There is potential to reduce the number of AWG channels further if needed.

The concept of using multiple different excitation wavelengths can be applied to various external target and different analytes. This way a smaller number of AWG channels suffices and the spectral range which can effectively be covered by the AWG can be extended. The sensor module has the advantages of improved compactness, reduced size and small costs compared to current solutions, which are still of the size of table top optical benches. The following discussion provides estimates of performance parameters of the proposed sensor module.

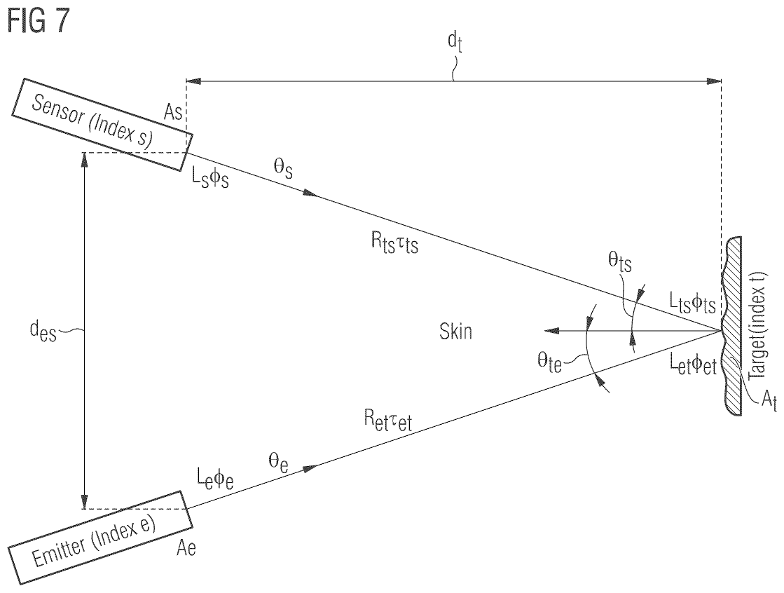

FIG. 7 shows an example scheme of illumination geometry. Emitter and detectors may not necessarily be perpendicular to a main surface of the module, but rather be tilted as shown in drawing. In this scheme the external target t is assumed to be the upper blood net dermis reflecting diffusely with a net reflectivity ρ. Pairs of emitter and detector have aperture distance of dₑₛ. Emitter and detector are aligned with the line of sight to the target and their exit and entrance optics are in touch contact with the skin surface with negligible air gap in between. Then with the terms defined in the drawing, the radiometric flux at sensor aperture is given in the form of the so called range equation as:

$${Φ}_{d} = \frac{ρ {A}_{s} {A}_{t} {Φ}_{e} {τ}_{e t} {τ}_{s} {τ}_{t s} cos ( {θ}_{t e} ) cos ( {θ}_{t s} )}{π {Ω}_{e} {R}_{e t}^{2} {R}_{t s}^{2}}$$

In the equation the terms are defined as follows. ρ is the effective target reflectivity (including the Raman conversion efficiency), Aₛ, Aₜ are sensor's aperture and target FOV area respectively, ϕₑ is the optical power of the emitter. τₑₜ and τₜₛ are the transmittivity of the medium between emitter-target, and target sensor respectively. τₛ describes the optical losses within the sensor up to the detector sub-element. θₜₑ and Ots describe the angles between emitter and sensor and the target surface normal respectively. Ωₑ is the emission angle of the emitter source and Rₑₜ_and Rₜₛ are the distances between emitter/sensor and the target respectively.

The size of the proposed sensor module for Raman spectroscopy depends on the size of the dispersive element. For example, the more channels added the larger the element gets. The module may have a total footprint size (e.g., about 1 cm²) is mainly determined by the size of the dispersive element and is a compromise between these parameters. Furthermore, the optical signal per channel is a fraction of the incoming optical power into the dispersive element divided by the number of channels. Thus there may be an upper limit to the number of channels determined by the signal-to-noise ratio of the detector element.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Furthermore, as used herein, the term “comprising” does not exclude other elements. In addition, as used herein, the article “a” is intended to include one or more than one component or element, and is not limited to be construed as meaning only one.

This patent application claims the priority of German patent application 102022117346.1, the disclosure content of which is hereby incorporated by reference.

### REFERENCES

10 housing
11 carrier
12 first chamber
13 second chamber
14 wall
15 emitter section
16 spectrometer section
17 aperture
18 aperture
30 light emitter arrangement
31 light emitter array
40 dispersive element
41 input port
42 output port
43 input side
44 output side
45 free space propagation region
46 grating waveguides
50 light detector arrangement
51 array of semiconductor light detectors

1. A sensor module for Raman spectroscopy, comprising a sensor package enclosing a light emitter arrangement, a dispersive element and a light detector arrangement comprising a light detector array, a comparator and lock-in amplifier arranged on or integrated into a carrier, wherein:
the light emitter arrangement is operable to emit light with multiple excitation wavelengths out of the sensor module,
the dispersive element is operable to receive light incident on the sensor module and operable to disperse the incident light into spectral components,
the light detector arrangement is operable to generate spectral sensor signals indicative of the spectral components, and
the comparator and lock-in amplifier is operable to receive spectral output signals of light detectors of the light detector array and to extract the spectral sensor signal.

2. The sensor module according to claim 1, wherein the dispersive element is operable to disperse the incident light into spatially separate spectral components.

3. The sensor module according to claim 1, wherein the dispersive element comprises:
an arrayed waveguide grating,
a diffraction grating,
a refractive prism, and/or
a poled domain prism.

4. The sensor module according to claim 1, wherein the light emitter arrangement comprises two or more light emitters, each operable to emit light out of the sensor module with an excitation wavelength from the multiple excitation wavelengths.

5. The sensor module according to claim 1, wherein the light emitter arrangement comprises at least one tuneable light emitter operable to emit light out of the sensor module to be tuned to an excitation wavelength from the multiple excitation wavelengths.

6. The sensor module according to claim 1, wherein each light detector of the light detector array is operable to generate one of the spectral sensor signal indicative of a respective spectral component.

7. The sensor module according to claim 1, wherein the carrier comprises a photonic integrated circuit.

8. The sensor module according to claim 7, wherein the photonic integrated circuit comprises:
a single input port to receive the incident light, the input port being an input waveguide,
at least one output port, the output port being an output waveguide, wherein the dispersive element is arranged between the input waveguide and the output waveguide on the photonic integrated circuit chip, wherein the output port is operable to couple the spectral components from dispersive element to the light detector arrangement.

9. The sensor module according to claim 8, wherein the input port comprises:
a grating fabricated on a surface of the photonic integrated circuit,
a tapered waveguide at the edge of the carrier,
a refractive lens,
a diffractive lens, and/or
an optical fiber.

10. The sensor module according to claim 8, wherein the output port comprises:
an array of waveguides, each waveguide located at specific spatial locations to couple the spectral components from dispersive element,
a single waveguide,
multiple waveguides with different widths, and/or
multiple waveguides with a specific spacing between individual waveguides.

11. The sensor module according to claim 1, wherein:
the sensor package comprises a hollow housing,
the housing comprises a first aperture to allow the light emitted by the light emitter arrangement to leave the sensor module,
the housing comprises a second aperture to allow incident light to enter the sensor module.

12. The sensor module according to claim 11, wherein
the housing comprises optically isolated first and second chambers,
the first chamber encloses the light emitter arrangement, and
the second chamber encloses the light detector arrangement.

13. The sensor module according to claim 1, wherein the module fits into a footprint of about 1 cm².

14. An electronic device comprising a sensor module for Raman spectroscopy according to claim 1 and a host system, wherein:
the sensor module is embedded in and electrically connected to the host system,
the host system comprises one of a mobile device, Smartphone, handheld computer, Smart Watch, handheld Medical-device, or a point-of-care device.

15. A method of conducting Raman spectroscopy, using a sensor module according to claim 1, the method comprising the steps of:
using the light emitter arrangement, emitting light with multiple excitation wavelengths out of the sensor module,
using the dispersive element, receiving light incident on the sensor module and dispersing the incident light into spectral components, and
using the light detector arrangement, generating spectral sensor signals indicative of the spectral components.
